(19) United States
(12) Patent Application Publication
(10) Pub. No.: US 2005/0148787 A1
(43) Pub. Date: Jul. 7, 2005

(54) Process for the preparation of propylene carbonate

(76) Inventors: Johannes Gerhardus Joseph Beckers, Amsterdam (NL); Evert Van Der Heide, Amsterdam (NL); Gerardus Martinus Maria Van Kessel, Amsterdam (NL); Jean-Paul Lange, Amsterdam (NL)

Correspondence Address: Jennifer D. Adamson;Shell Oil Company, Legal-Intellectual Property, P.O. Box 2463, Houston, TX, 77252-2463, US

(21) Appl. No.: 10/994,741

(22) Filed: Nov. 22, 2004

(30) Foreign Application Priority Data
Nov. 26, 2003 (EP) 03257450.1

(51) Int. Cl. C07C 69/96

(52) U.S. Cl. 558/260

(57) ABSTRACT

The invention is related to a process involving contacting a propylene oxide feed with carbon dioxide in the presence of a suitable catalyst to obtain a reaction mixture containing propylene carbonate in which process the propylene oxide feed contains at least 50 ppmw of acids and/or carbonyls.

### FIELD OF THE INVENTION

The present invention relates to a process comprising contacting a propylene oxide feed with carbon dioxide in the presence of a suitable catalyst to obtain a reaction mixture comprising propylene carbonate.

### BACKGROUND OF THE INVENTION

It is known to convert alkylene oxide into a cyclic alkylene carbonate in the presence of a suitable catalyst. Such process has been described, for example, in U.S. Pat. No. 6,258,962. The cyclic alkylene carbonate may be converted further through reaction with an alcohol such as methanol to give dimethylcarbonate and a diol. Another option is to react the cyclic alkylene carbonate with water to obtain a diol and carbon dioxide. Diols such as 1,2-ethanediol and 1,2 propanediol and dimethylcarbonate are widely used in the chemical industry.

WO-A-03/000641 describes a process for preparing dialkyl carbonates and diols from alkylene oxides. It is mentioned in WO-A-03/000641 that the alkylene oxide feed may contain various impurities. As an example it is indicated that ethylene oxide may contain carbon dioxide, water and aldehydes. No specific amounts or concentrations are indicated, but as explained below, amounts of less than 50 ppmw of aldehydes are expected.

Alkylene oxide is usually produced in a process comprising (i) reacting alkenes with suitable oxidant to yield a reaction mixture containing alkylene oxide, (ii) separating wet crude alkylene oxide from the reaction mixture obtained in step (i), and optionally (iii) removing water from the wet crude alkylene oxide by at least one distillation treatment to obtain dry crude alkylene oxide. Step (ii) generally consists of (iia) removing unreacted alkene from the reaction mixture, and (iib) separating the wet crude alkylene oxide from the mixture obtained in step (iia) by at least one distillation treatment. The thus obtained wet or dry crude alkylene oxide, further referred to herein as crude alkylene oxide, still contains minor quantities of by-products having a boiling point close to the alkylene oxides and/or forming azeotropic mixtures with the alkylene oxide. Examples of such by-products are acids and carbonyls (such as aldehydes and ketones, for example).

The presence of impurities stemming from the manufacture of alkylene oxide derivatives is generally considered undesirable. Someone skilled in the art would expect that the contaminants present in the alkylene oxide would have a negative impact on the catalyst used in the process to prepare cyclic alkylene carbonates, especially if the catalyst is a homogeneous catalyst, more especially if it is a phosphonium halide catalyst.

Therefore, the crude alkylene oxide obtained from step (ii) or optionally step (iii) is submitted to an additional purification treatment (iv).

Only substantially purified alkylene oxide (further referred to herein as pure alkylene oxide) is used in the preparation of cyclic alkene carbonates. Pure alkylene oxide as commercially available will have an alkylene oxide content of more than 99.95% by weight and a total amount of acids and carbonyls of less than 50 ppmw (parts per million by weight).

It would be useful to improve the catalyst stability.

### SUMMARY OF THE INVENTION

The present invention is directed to a process comprising contacting a propylene oxide feed with carbon dioxide in the presence of a suitable catalyst to obtain a reaction mixture comprising propylene carbonate in which process the propylene oxide feed comprises at least 50 ppmw of acids and/or carbonyls.

### DETAILED DESCRIPTION OF THE INVENTION

Surprisingly it has now been found, contrary to expectation, that less catalyst decomposition was observed if a propylene oxide feed according to the present invention was used. Without wishing to be bound by any theory, it is thought that the improved stability is due to the presence of acids and carbonyls in the feed according to the present invention.

Moreover, it has been found that crude propylene oxide may be advantageously used in such a process for preparation of cyclic alkylene carbonates.

The carbon dioxide for use in the present process may be either pure carbon dioxide or carbon dioxide containing further compounds. Carbon dioxide which is especially suitable for use in the present invention is carbon dioxide which has been separated off in subsequent steps of the present process. Carbon dioxide may either be separated off directly after the propylene oxide has reacted with carbon dioxide or at a later stage. The extent to which carbon dioxide is purified depends on the nature and the amounts of contaminants present in the carbon dioxide.

The propylene oxide feed is reacted with carbon dioxide at suitable operating conditions. Such process conditions will generally comprise a temperature of from 50° C. to 200° C., more specifically 100° C. to 150° C., and a pressure of at least 5×10⁵ N/m², more specifically a pressure of from 5 to 100×10⁵ N/m², most specifically of from 10 to 30×10⁵ N/m².

The catalyst for use in the present invention generally will be a homogeneous catalyst. A specific catalyst which is known to be suitable is a homogeneous phosphorus containing catalyst. The phosphorus will usually not be present in its elemental form in the catalyst. Phosphorus containing compounds which are suitable catalysts are phosphonium compounds. The catalyst preferably is a homogeneous phosphonium catalyst, more specifically a phosphonium halide catalyst. Tetraalkylphosphonium halide catalysts, more specifically a tributyl-methyl phosphonium iodide, were found to be suitable for use in the present invention.

A catalyst which was found to be especially advantageous was a phosphonium bromide catalyst. The phosphonium bromide catalyst preferably is a tetraalkyl phosphonium bromide catalyst of the formula R¹R²R³R⁴PBr (I) in which the groups R¹, R², R³ and R⁴ each independently represent an alkyl group having of from 1 to 10 carbon atoms, more specifically of from 2 to 6 carbon atoms. Preferably, R¹, R², R³ and R⁴ each represent n-butyl.

The catalyst may be either added as such or may be formed in-situ.

The catalyst may be added to the reactor as a solution of the catalyst in an inert solvent such as in a cyclic carbonate. The catalyst may be added either to the propylene oxide or to the carbon dioxide or to the mixture of both. Preferably, the catalyst solution is added to the mixture of propylene oxide and carbon dioxide.

The propylene oxide feed for use in the present process may be prepared according to steps (i) to (iii). As water may be present in the process according to the present invention, the propylene oxide feed preferably is prepared by the steps of: (i) reacting propene with a suitable oxidant to yield a reaction mixture containing propylene oxide, and (ii) separating propylene oxide from the reaction mixture obtained in (i).

In step (i), propene is reacted with a suitable oxidant. Suitable oxidants are capable of epoxidation of propene to the corresponding propylene oxide. The oxidants include oxygen, and oxygen-containing gases or mixtures such as air and nitrous oxide. Other suitable oxidants are hydroperoxide compounds, such as hydrogen peroxide and aromatic or aliphatic hydroperoxides. The hydroperoxide compounds preferably include hydrogen peroxide, tertiary butyl hydroperoxide, ethyl benzene hydroperoxide and isopropyl benzene hydroperoxide. Ethyl benzene hydroperoxide is most preferred. Even more preferably the process is an integrated styrene monomer/propylene oxide process, as for instance described in U.S. Pat. No. 6,504,038, which is herein incorporated by reference.

The propylene oxide feed for use in the present invention may be separated from the reaction mixture obtained. Although such separation may be carried out in any way know to someone skilled in the art, the separation will generally comprise (iia) removing unreacted alkene from the reaction mixture obtained in (i), and (iib) separating crude propylene oxide from the mixture obtained in step (iia) by at least one distillation treatment. This set-up makes it possible to reduce the size of the distillation unit of step (iib) while a high throughput is maintained.

The first distillation of the reaction mixture containing the propylene oxide (iia) gives an overhead fraction containing unreacted alkene and some low boiling impurities. The distillation treatment may be carried out at a pressure of from 1 to 20×10⁵ N/m² (bar) and at a temperature in the range of from 10° C. to 250° C. The distillation may remove the unreacted alkenes along with other low boiling impurities. In step (iib), crude propylene oxide is generally removed together with lower boiling contaminants as an overhead product from the reaction mixture obtained in step (iia). The distillation treatment of (iib) may be carried out at a pressure of from 0.1 to 20×10⁵ N/m², and at a temperature in the range of from 0° C. to 250° C. Preferably, this distillation treatment is carried out at a pressure in the range of from 0.1 to 1×10⁵ N/m², and at a temperature in the range of from 10° C. to 200° C.

The propylene oxide obtained in step (ii) will generally still contain a significant amount of water, specifically from 50 ppmw to 5000 ppmw (parts per million by weight), more specifically from 100 ppmw to 4800 ppmw of water. The amount of water present in the propylene oxide obtained from step (ii) more specifically contains at most 4500 ppmw, more specifically at most 4000 ppmw, yet more specifically at most 3500 ppmw, and most specifically at most 3000 ppmw of water.

In an optional step (iii), part of the water still present in the propylene oxide may be removed as an overhead product from the crude propylene oxide, as for instance described in U.S. Pat. No. 3,607,669, which is herein incorporated by reference. In at least one distillation treatment of step (iii), one or more entrainer components may be added to the propylene oxide. Entrainer components tend to reduce the amount of components other than propylene oxide in the bottom product of the distillation unit, in particular water. Preferred entrainer components are aliphatic hydrocarbons having 4 or 5 carbon atoms.

This distillation treatment of step (iii) may be carried out at a pressure of from 1 to 20×10⁵ N/m², and at a temperature range of from 0° C. to 200° C. Preferably, the distillation treatment is carried out at a pressure in the range of from 5 to 10×10⁵ N/m², and at a temperature in the range of from 10° C. to 150° C. The propylene oxide obtained from step (iii) generally contains from 0 ppmw to 150 ppmw of water, more specifically from 10 ppmw to 150 ppmw of water.

Whereas the separation of the unreacted alkenes and part of the water could be effected without difficulty, as described in steps (iia), (iib) and (iii), the separation of by-products such as aldehydes and acids from the propylene oxide is particularly difficult, even by fractional distillation.

Distillation units used for step (iib) and optionally (iia) and (iii) do not separate the propylene oxides from close boiling contaminants. Such separation would require columns having a large number of trays and hence strongly limit the throughput.

When only step (ii) and optional step (iii) described above are used, a pure propylene oxide having an propylene content of more than 99.95% by weight and less than 50 ppmw acids and carbonyls, is not be obtained, as these process steps (ii) and (iii) have insufficient separation capacity and would give unacceptable loss of alkylene oxide. Therefore prior art processes applied an additional purification (iv). Such an additional purification (iv) usually comprises multiple process steps as the removal of impurities stemming from step (i) is particularly difficult. This additional purification requires complex equipment and consumes large amounts of energy. This has been described in EP-A-0,755,716, U.S. Pat. No. 3,578,568, and WO 02/070497. Additionally, these further processing steps may generate poly(alkylene oxide) of high molecular weight in the purified alkylene oxide. It will be clear that the presence of such high molecular weight polymers is undesirable in the process of the present invention. Therefore, alkylene oxide has to be treated to remove not only the impurities originating from its manufacture but also to remove impurities that are generated during the purification treatment itself. Advantageously it has now been found that such a purification is no longer needed and crude propylene oxide prepared by step (ii) and optionally step (iii) may be used directly in the process according to the invention.

Preferably, the crude propylene oxide feed comprises on total composition from 95.00% by weight to 99.95% by weight of propylene oxide, and of from 5.0% by weight to 0.05% by weight of compounds other than propylene oxide. The crude propylene oxide preferably comprises at least 96.00% by weight of propylene oxide, more preferably more than 96.00% by weight, even more preferably at least 97.00% by weight, more preferably more than 97.00% by weight, even more preferably at least 99.00% by weight, again more preferably more than 99.00% by weight, and most preferably at least 99.50% by weight of propylene oxide. Preferably, the crude propylene oxide comprises at most 99.93% by weight of propylene oxide, more preferably less than 99.90% by weight, again more preferably at most 99.85% by weight, yet more preferably less than 99.83% by weight, again more preferably at most 99.80% by weight, more preferably less than 99.80% by weight, yet more preferably at most 99.79% by weight, and most preferably at most 99.78% by weight of propylene oxide, the remainder being compounds originating from the epoxidation reaction of step (i), or reaction products of these compounds during steps (ii) and/or (iii).

The compounds which are present in the propylene oxide feed besides the propylene oxide itself generally are alkenes, alkanes and oxygen containing by-products such as aldehydes, ketones, alcohols, ethers, acids and esters. Specific compounds which were found to be present were water, acetone, acetaldehyde, propionaldehyde, acetic acid, formic acid and methanol. The propylene oxide feed for use in the present invention will preferably comprise a total amount of acids and carbonyls in an amount of from 50 to 10000 part per million by weight (ppmw). More preferably, the total amount of acids and carbonyls is at least 200 ppmw, more specifically at least 300 ppmw, most specifically at least 500 ppmw. More preferably the total amount of acids and carbonyls is mainly made up from carbonyls. Hence, more preferably a propylene oxide feed is used which comprises at least 50 ppmw carbonyls. More preferably the feed comprises at least 100 ppmw carbonyls, even more preferably at least 200 ppmw, and yet more preferably at least 300 ppmw carbonyls. In a further preference, the feed comprises an amount of carbonyls in the range from 200 to 10000 ppmw carbonyls, and more preferably even in the range from 300 to 5000 ppmw carbonyls. Preferred carbonyls are acetone, acetaldehyde and propionaldehyde.

The propylene oxide feed may also comprise a small quantity of poly(propylene oxide) having a weight average molecular weight of more than 2000. The amount of poly(propylene oxide) in the propylene oxide feed preferably is less than 50 ppmw. The crude propylene oxide more preferably contains at most 30 ppmw, yet more preferably at most 20 ppmw particularly more preferably at most 15 ppmw, again more preferably at most 12 ppmw, yet more preferably at most 5 ppmw, and most preferably contains at most 3 ppmw of poly(propylene oxide) having a weight average molecular weight of more than 2000.

The propylene oxide feed may contain crude propylene oxide in combination with pure propylene oxide. Pure propylene oxide comprises on total composition more than 99.95% by weight of propylene oxide. Preferably, pure propylene oxide contains a total amount of acids and carbonyls of less than 100 ppmw, preferably less than 80 ppmw, and most preferably less than 50 ppmw.

The reaction mixture obtained by the present invention preferably is used in the manufacture of 1,2-propanediol and optionally dimethylcarbonate. Such process generally comprises the steps of (a) contacting a propylene oxide feed with carbon dioxide in the presence of a suitable catalyst according to the present invention to obtain a reaction mixture comprising propylene carbonate, (b) optionally removing unreacted carbon dioxide from the reaction mixture obtained in step (a), (c) contacting the propylene carbonate containing reaction mixture with water and/or methanol in the presence of a suitable catalyst to obtain 1,2-propanediol and optionally dimethylcarbonate, and (d) separating 1,2-propanediol from the reaction product obtained in step (c).

This process for preparing 1,2-propanediol and optionally dimethylcarbonate may comprise further process steps for removing specific compounds from the reaction mixture besides the optional removal of carbon dioxide in step (b). Step (b) has the advantage that it may substantially reduce the volume of the reaction mixture to be subjected to step (c). Further purification steps depend on the actual process conditions and will be obvious to someone skilled in the art. A further purification step may comprise separation of unreacted propylene oxide if the conversion levels are very low.

If a homogeneous catalyst is used in step (a) of the present invention, it may be advantageous to leave this homogeneous catalyst in the reaction mixture while subjecting it to further processing steps. This has been found to be especially advantageous if the catalyst is a homogeneous phosphorus containing catalyst.

Water and/or an alcohol is added to the reaction mixture comprising propylene carbonate. The alcohol used may comprise one or two alcohol groups. Preferably, the alcohol is non-aromatic and is chosen from the group consisting of C₁-C₅ alkyl alcohols. Preferably, the alcohol is methanol and/or ethanol. Most preferably, the alcohol is methanol.

Preferably, either solely water or solely alcohol is added to the reaction product containing propylene carbonate and phosphorus containing catalyst. It is preferred to add water only.

The catalysts for use in step (c) are well known in the art. The catalyst preferably is a heterogeneous catalyst especially if the propylene carbonate is contacted with water only. Examples of such heterogeneous catalysts comprise solid inorganic compounds such as alumina, silica-alumina, alumina carrying a copper compound, silica-alumina carrying a copper compound, silica-magnesia, aluminosilicate, gallium silicate, zeolites, metal-exchanged zeolites, ammonium-exchanged zeolites, zinc on a support, lanthanum on a support, a mixture of aluminium and magnesium (hydr)oxide and ion-exchange resins.

Preferably, the catalyst employed in step (c) is chosen from the group consisting of a mixture of aluminium and magnesium (hydr)oxide, zinc on a support, lanthanum on a support and alumina. These catalysts will be described hereinafter in more detail. Most preferably, the catalyst is alumina.

The mixture of aluminium and magnesium (hydr)oxide preferably has a magnesium to aluminium molar ratio in the range of from above 4 to 50, more preferably of from above 4 to 20. In the preparation of the catalyst, generally a so-called mixed magnesium/aluminium hydroxide is prepared. However, it might be that under working conditions mixed magnesium/aluminium oxides and/or carbonates are present. Our reference to a mixture of aluminium and magnesium (hydr)oxide covers both mixtures of aluminium and magnesium hydroxide and mixtures of aluminium and magnesium oxide and a combination of both mixtures. These mixtures were found to give the highest activity at a molar ratio of from 5 to 15, most specifically of from 5 to 10. Preferred catalysts are described in PCT patent application PCT/EP02/12640.

In another preferred embodiment of the present invention, the catalyst comprises a lanthanum compound on a support. A preferred catalyst comprises at least 7% wt of lanthanum supported on a support. The lanthanum compound preferably is La₂O₃ or a precursor thereof. Under reaction conditions this lanthanum compound may be temporarily and/or reversibly converted due to the reaction conditions into lanthanum hydroxide (La(OH)₃), lanthanumoxyhydroxide (LaO(OH)) and/or corresponding alcoholate species such as (La(OR)₃ or LaO(OR)).

As a support for the lanthanum containing catalyst any suitable support may be used. The support preferably is substantially inert under the reaction conditions and is provided with sufficient mechanical strength. Potential supports comprise clay minerals, inorganic supports such as Al₂O₃, SiO₂, MgO, TiO₂, ZrO₂, ZnO and mixtures thereof. Other examples are a kaolinite, a hallosyte, a chrysotile, a montmorillonite, a beidellite, a hectorite, a sauconite, a muscovite, a phlogopite, a biotite, a hydrotalcite and talc. Particularly preferred are the inorganic supports selected from the group consisting of A1₂O3, SiO₂, MgO, TiO₂, ZrO₂, ZnO and mixtures thereof.

The lanthanum containing catalyst comprises preferably in the range of from 7 wt. % to 40 wt. % of lanthanum based on total amount of catalyst. The lanthanum containing catalyst may be produced using any suitable method. A preferred method comprises impregnating a support with a lanthanum containing salt, and subsequently drying and calcining the impregnated support. After impregnation the impregnated support may be dried and subsequently calcined. Calcination is generally carried out at a calcination temperature from between 120° C. to 700° C. The catalyst activity may be increased even further if the catalyst is calcined at a temperature in the range of from 350° C. to 600° C. Preferred catalysts are described in PCT patent application PCT/EP02/12638.

A further catalyst which is especially suitable for use in step (c) of the present invention is a zinc supported catalyst. The support preferably is selected from the group consisting of Al₂O₃, SiO₂, MgO, TiO₂, ZrO₂, Cr₂O₃, carbon and mixtures thereof. The zinc supported catalyst may be prepared by impregnation of silica, alumina or mixtures of aluminium and magnesium (hydr)oxide with a zinc nitrate solution. Preferably, the zinc supported catalysts comprise at least 15% wt of zinc on a support having a surface area of at least 20 m²/g, more preferably at least 40 m²/g. Preferred catalysts are described in the patent applications claiming priority of European patent application 02256347.2.

A further catalyst which is preferably used is a catalyst consisting of alumina. Preferably, the alumina is gamma-alumina.

If solely water is added to the reaction product containing the propylene carbonate, the process is preferably carried out at a temperature of from 50 to 300° C., preferably of from 80 to 250° C., more specifically of from 100 to 200° C. The pressure may vary widely, and preferably is at most 50×10⁵ N/m², more specifically at most 20×10⁵ N/m².

If solely alcohol, more specifically methanol, is added to the reaction product containing the propylene carbonate, the process is preferably carried out at a temperature of from 50 to 300° C., more preferably of from 100 to 200° C. The pressure preferably is of from 1 to 100×10⁵ N/m², preferably of from 5 to 60×10⁵ N/m², more specifically of from 20 to 40×10⁵ N/m².

The 1,2-propanediol may be separated from the reaction product obtained in step (c) in any way known in the art. A further option is to combine steps (c) and (d) by using a catalytic distillation.

A preferred separation step (d) comprises distillation of the reaction product obtained in step (c). One or more of the fractions separated will have a high content of 1,2-propanediol. 1,2-Propanediol obtained by distillation will usually be sufficiently pure for use as such. If required, small amounts of by-products may be removed separately. A well known by-product in the manufacture of 1,2-propanediol is dipropylene glycol. The latter may be removed relatively easily by distillation.

If an alcohol is added in step (c), dialkylcarbonates such as dimethylcarbonate will be present in the reaction product of step (c). In such process, the process preferably further comprises separating the dimethylcarbonate from the reaction product in step (d). The dimethylcarbonate may be separated off in any way known to be suitable to someone skilled in the art. Preferably, the dimethylcarbonate is separated by distillation.

If homogeneous catalyst is present in the crude reaction product of step (c), this catalyst is preferably separated off from the reaction product obtained in step (c) and/or step (d). The catalyst obtained may be recycled for use in step (a). The catalyst may be recycled in combination with further compounds either added to or formed in the process according to the present invention. Preferably, the catalyst will be recycled while being dissolved in 1,2-propanediol.

Surprisingly, it was found that the presence of a solvent may be advantageous in the process according to the present invention. A protic solvent was found to reduce decomposition of the phosphorus containing catalyst. 1,2-Propanediol was found to be an especially advantageous solvent. The solvent is preferably present during the whole process such as in the conversion steps (a) and/or (c) and separation steps (b) and/or (d). However, water and/or alcohol is present in steps (c) and (d) while additionally 1,2-propanediol is either being formed or is present in these steps. Therefore, it generally suffices to add protic solvent, preferably 1,2-propanediol, to step (a). The solvent is then present in the subsequent steps. Most preferably, the protic solvent is combined with the phosphorus containing catalyst before being added to step (a).

The present invention is further illustrated by the following examples. These examples are given for further illustration of the invention and are not limiting the invention.

### EXAMPLE

The experiments were carried out in a 60 ml Hastelloy C (Hastelloy is a trademark of Haynes International, Inc.) autoclave reactor equipped with a heating jacket and a gas inlet, and stirred by means of a gas-dispersing propeller.

120 grams of propylene oxide feed was added to the reactor. The propylene oxide feed comprised 99.80% by weight of propylene oxide, 1400 ppmw of propionaldehyde and 50 ppmw of water. The remainder consisted of impurities such as acids and alkenes.

The reactor was then sealed and carbon dioxide (CO₂) was introduced to a total pressure of 20×10⁵ N/m² (bar). The reactor was heated to 150° C. under stirring. At 150° C., a solution of 5 grams 1,2-propanediol and 0.3 gram tetrabutylphosphonium bromide was injected into the reactor and the injection line was flushed with 9 grams of 1,2-propanediol. After 5 hours at the above-described conditions, the reactor was cooled down rapidly, allowed to decompress, and samples were taken.

The amount of propylene carbonate obtained was determined by gas chromatography. Catalyst decomposition to the corresponding phosphine oxide was determined using 31P-NMR.

It was found that 190 grams of propylene carbonate was obtained. Further, it was found that 0.012 grams of tributylphosphineoxide was formed.

### Comparative Example

Example 1 was repeated but using as propylene oxide feed a purified propylene oxide with a purity of more than 99.98% by weight containing 15 ppmw of propionaldehyde, 15 ppmw of acetaldehyde and 50 ppmw of water.

It was found that 190 grams of propylene carbonate was obtained. Further, it was found that 0.022 grams of tributylphosphineoxide was formed.

1. A process comprising contacting a propylene oxide feed with carbon dioxide in the presence of a suitable catalyst to obtain a reaction mixture comprising propylene carbonate in which process the propylene oxide feed comprises at least 50 ppmw of acids and/or carbonyls.

2. The process of claim 1, in which process the catalyst is a homogeneous catalyst.

3. The process of claim 1, in which the propylene oxide feed comprises at least 50 ppmw of carbonyls.

4. The process of claim 2, in which the propylene oxide feed comprises from 95.00% by weight to 99.95% by weight of propylene oxide.

5. The process of claim 2, in which process the catalyst is a homogeneous catalyst.

6. The process of claim 2, in which the propylene oxide feed is obtained by the steps of:
(i) reacting propene with a suitable oxidant to yield a reaction mixture containing propylene oxide; and,
(ii) separating propylene oxide from the reaction mixture obtained in (i).

7. The process of claim 1, in which the propylene oxide feed comprises from 95.00% by weight to 99.95% by weight of propylene oxide.

8. The process of claim 1, in which the propylene oxide feed is obtained by the steps of:
(i) reacting propene with a suitable oxidant to yield a reaction mixture containing propylene oxide; and,
(ii) separating propylene oxide from the reaction mixture obtained in (i).

9. A process according to claim 8, wherein the propylene oxide feed obtained in step (ii) comprises of from 50 to 5000 ppmw of water, based on total composition.

10. The process of claim 8, in which process the catalyst is a homogeneous catalyst.

11. The process of claim 10, in which process the catalyst is a tetraalkyl phosphonium bromide catalyst of the formula R¹R²R³R⁴PBr (I) in which each R¹, R², R³ and R⁴ each independently represent an alkyl group having of from 1 to 10 carbon atoms.

12. The process of claim 11, wherein R¹, R², R³ and R⁴ in formula (I) each represent n-butyl.

13. A process for the preparation of 1,2-propanediol and optionally dimethylcarbonate, which process comprises the steps of:
(a) contacting a propylene oxide feed with carbon dioxide in the presence of a suitable catalyst to obtain a reaction mixture comprising propylene carbonate;
(b) optionally removing unreacted carbon dioxide from the reaction mixture obtained in step (a);
(c) contacting the propylene carbonate containing reaction mixture with water and/or methanol in the presence of a suitable catalyst to obtain 1,2-propanediol and optionally dimethylcarbonate; and,
(d) separating 1,2-propanediol from the reaction product obtained.

14. The process of claim 11, in which in step (a) the propylene oxide feed comprises at least 50 ppmw of carbonyls.

15. The process of claim 11, in which in step (a) the propylene oxide feed comprises from 95.00% by weight to 99.95% by weight of propylene oxide.

16. The process of claim 11, in which in step (a) the propylene oxide feed is obtained by the steps of:
(i) reacting propene with a suitable oxidant to yield a reaction mixture containing propylene oxide; and,
(ii) separating propylene oxide from the reaction mixture obtained in (i).

17. The process of claim 11, in which process the catalyst is a homogeneous catalyst.

18. The process of claim 17, in which process the catalyst in step (a) is a tetraalkyl phosphonium bromide catalyst of the formula R¹R²R³R⁴PBr (I) in which each R¹, R², R³ and R⁴ each independently represent an alkyl group having of from 1 to 10 carbon atoms.
